(19) United States
(12) Patent Application Publication
(10) Pub. No.: US 2015/0265781 A1
(43) Pub. Date: Sep. 24, 2015

(54) COMPACT FLUID WARMER

(71) Applicant: BOARD OF REGENTS THE UNIVERSITY OF TEXAS SYSTEM, Austin,, TX (US)

(72) Inventors: Brijesh S. Gill, Houston, TX (US); Kevin Aroom, Houston, TX (US); Charles Cox, JR., Bellaire, TX (US)

(21) Appl. No.: 14/437,106

(22) PCT Filed: Oct. 22, 2013
(86) PCT No.: PCT/US13/66150

§ 371 Date: Apr. 20, 2015

Related U.S. Application Data
(60) Provisional application No. 61/716,752, filed on Oct. 22, 2012.

(51) Int. Cl. A61M 5/44

(52) U.S. Cl. CPC A61M 5/445; A61M 2205/10; A61M 2205/362

(57) ABSTRACT

A portable apparatus to warm a stream of biocompatible fluid prior to introduction into a patient comprises a heat exchanger base with a first side and a second side, a gas chamber cover coupled to the first side to form a gas chamber therebetween, a fluid warming chamber cover coupled to the second side to form a fluid warming chamber therebetween, an air-fuel mixing chamber with an outlet feeding an inlet to the gas chamber, a catalyst member disposed within a catalyst compartment of the gas chamber to receive an air-fuel mixture from the inlet to the gas chamber, a tortuous pathway between the catalyst compartment and an exhaust port of the gas chamber, an air mover to receive ambient air and discharge air into the air-fuel mixing chamber, and a fuel storage tank connected to feed a stream of fuel gas to the air-fuel mixing chamber.

### STATEMENT OF RELATED APPLICATIONS

This application depends from and claims priority to PCT/US2013/066150 entitled Compact Fluid Warmer filed on Oct. 22, 2013, which depends from and claims priority to U.S. Provisional Application No.: 61/716,752 filed on Oct. 22, 2012.

### STATEMENT OF FEDERALLY SPONSORED RESEARCH & DEVELOPMENT

This invention was made with United States Government support under Grant No. W81XWH-10-1-01060 awarded by the United States Department of Defense. The United States government has certain rights in the invention.

### FIELD OF THE INVENTION

The present invention relates to a portable apparatus for warming biocompatible fluids for use in the treatment of patients. The invention may be used to warm intravenous fluids for trauma resuscitation or to warm air from a ventilator circuit A compact nature of the fluid warmer makes it particularly well suited for field applications, such as surgical hospitals near a combat zone.

### BACKGROUND OF THE INVENTION

Hypothermia is quite common in injured patients, including patients experiencing trauma. Hypothermia produces a number of physiologic derangements which worsen the effects of major injury. Several relevant enzyme systems begin to lose efficiency as their ambient temperature falls. For example, the myocardium, which is dependent on the function of membrane-channel type enzymes for normal electrical function, shows a predictable series of atrial followed by ventricular arrhythmias as core temperature falls below 34° C. Cardiac output is further compromised by poor function of intrinsic myocardial components, with bovine myocardium showing a linear decrease in developed tension with decreasing temperature.

Hypothermia also exacerbates hemorrhagic shock in multiple ways. The onset of coagulopathy, which accompanies hypothermia, has been shown to result from malfunction of both dotting factors and platelets. While profound hypothermia may be tolerated by immersion or cardiac surgery patients, the presence of hypothermia in trauma patients predicts significantly higher mortality. Mortality doubles for heterogeneous groups of trauma patients at 34° C., and survival after trauma is very rare when the core temperature falls below 32° C. This effect is greater for more severely injured patients.

The development of hypothermia conies from several factors. Body heat is convectively lost to the environment, and this effect is enhanced by bleeding or the presence of large surface area bums. The body loses both central thermoregulation and peripheral shivering response after traumatic injury. Less heat is produced peripherally as perfusion decreases in shock.

The administration of intravenous fluids is used in trauma resuscitation. The administration of fluid at ambient temperature, however, induces hypothermia. This condition is worse in more severely injured patients, who require the most fluid and have the least ability to tolerate the additional insult of decreased core temperature. Hypothermia and mortality clearly increase after the administration of five liters of crystalloid or five units of packed red blood cells, and the onset of hypothermia increases the incidence of coagulopathy in injured patients, particularly in the presence of acidosis.

As used herein, the phrase “biocompatible fluid” refers to a fluid that is appropriate for infusion into the human body including, but not limited to, normal saline and its less concentrated derivatives, Ringer's lactate, and hypertonic crystalloid solutions; blood and fractions of blood including plasma, platelets, albumin and cryoprecipitate; intravascular volume expanding blood substitutes including hetastarch, polymerized hemoglobin, perfluorocarbons; medications reconstituted with saline or sterile water; and medical gasses including air, oxygen, helium, nitric oxide, and combinations thereof.

Prior art methods of treating hypothermia include direct intravenous fluid warming. The fluid that is warmed may be the blood other biocompatible liquid. Prior art devices used to warm one or more biocompatible fluids for use in the treatment of trauma have used electricity as their heating source. These systems are referred to herein as “biocompatible liquid infusion systems.” Electrically heated biocompatible fluid infusion systems have several shortcomings. If the source of electrical energy is alternating current from a central generating station, the unit can then only be used in locations where such alternating current is available. This significantly limits the locations where the units may be used. Locations such as non-industrialized nations or battlefield locations are likely not have readily available sources of alternating current to power such systems. Batteries may be used to generate electrical energy. However, it is believed that sufficient power to heat a single liter of fluid to 20° C. within a ten-minute time period would require a rechargeable battery the size and weight of a large laptop computer. In such a case, the weight of the battery would exceed the weight of a liter of saline fluid. The size and weight of such a unit would severely limit its portability. Additionally, the battery would require recharging after each liter of biocompatible fluid is delivered.

Other conventional warming devices may use a venturi fuel-air mixer to mix fuel and air for being introduced into a catalytic combustion chamber. Heat is generated and transferred in a heat exchanger downstream from the combustion chamber, and exhaust gas is liberated at an exhaust port. This type of warming device entrains ambient air into the fuel flow stream to provide passive mixing of air with fuel prior to entry into a catalytic combustion chamber. The nature of the venturi requires a certain linear length of fuel pathway between the point, of fuel introduction and the point of fuel-air mixture discharge to allow for complete fuel and air mixing. If the length of the fuel pathway between the point of fuel introduction and the point of fuel-air mixture discharge is insufficient, the mixing of the fuel and air is incomplete and the mixture will not provide satisfactory combustion on the catalyst. The amount of heat generated will be insufficient to warm the heat exchanger and the exhaust gas discharge may contain unwanted carbon monoxide gas due to incomplete oxidation of the fuel component.

The present invention overcomes the limitations of prior art biocompatible fluid infusion systems h providing a biocompatible liquid infusion system that is not dependent upon electrical energy as a heat source, thereby enhancing portability and utility in field applications. The present invention is light enough and compact enough to be used in field hospital environments that are remotely located from large central hospitals and from sources of alternating electrical current. The present invention may also be used to warm air delivered to a patent via a ventilation circuit.

### SUMMARY OF THE INVENTION

An embodiment of an apparatus of the present invention comprises a gas flow chamber on a first side of the apparatus having an air-fuel mixture inlet, a catalyst compartment and at least one tortuous combustion products pathway originating at the catalyst compartment and terminating at an exhaust was port, a fluid warming chamber on a second side of the apparatus to conductively receive heat generated in the gas flow chamber and having, a fluid inlet connectable to a source of fluid, a fluid warming surface and a fluid outlet connectable to a patient, an air-fuel mixing chamber having an air inlet, a fuel port and an air-fuel mixture outlet, a motor-driven air mover having an air intake to receive ambient air and an air outlet disposed to discharge air to the air inlet of the air-fuel mixing chamber, and a fuel assembly comprising a fuel storage tank, a valve to receive a stream of fuel from the tank and a fuel port connector coupled to provide fuel from the valve to the fuel port of the air-fuel mixing chamber, wherein a stream of an air-fuel mixture emerging from the air-fuel mixing chamber enters the catalytic compartment containing the catalyst member and combusts to create a stream of heated combustion products, wherein the combustion products flow through the at least one tortuous pathway to the exhaust port where the combustion products are liberated to the atmosphere, and wherein a stream of fluid from the source of fluid enters the fluid warming, chamber through the fluid inlet, is warmed along, the warming surface and is removed from the fluid warming chamber through the fluid outlet. An embodiment of the apparatus may further comprise a catalyst member that comprises one of palladium and platinum. An embodiment of the apparatus may comprise a tank wherein the fuel stored in the tank is a hydrocarbon gas. An embodiment of the apparatus may further comprise a heat exchanger base comprising a metal alloy. An embodiment of the apparatus may comprise a heat exchanger base comprising, a conductive material such as stainless steel or, more preferably, aluminum due to its high conductivity and low density. An embodiment of the apparatus may be used to warm a fluid comprising one of blood and intravenous fluid. An embodiment of the apparatus may further comprise a fuel cell configured to receive a flow of fuel gas and to generate an electrical current to operate an electrically-powered motor within the air mover. An embodiment of the apparatus wherein the valve is adjustable to vary a rate of flow of fuel from the storage tank to the air-fuel mixing chamber. An embodiment of the apparatus may comprise a warming surface of the fluid warming chamber with an undulating surface to increase the surface area across which heat can be received from the gas chamber and transferred to the fluid within the fluid warming chamber.

Another embodiment of the apparatus comprises a heat exchanger base having a first side and a second side, a gas chamber cover securable to the first side of the heat exchanger base to form a gas chamber therebetween, the gas chamber having, an inlet, a catalyst compartment, a tortuous pathway and an exhaust port, a biocompatible fluid warming chamber cover securable to the second side of the heat exchanger base to form a biocompatible fluid warming chamber therebetween, the biocompatible fluid warming chamber having an inlet connectable to a source of biocompatible fluid, an outlet connectable to a patient, and a fluid warming, surface therebetween, an air-fuel mixing chamber having an outlet sealably engaging the inlet to the gas chamber, a catalyst member disposed within the catalyst compartment of the gas chamber, an air mover having an ambient air inlet and an air outlet sealably engaging, an air intake of the air-fuel mixing chamber, a storage tank containing a fuel, and a valve connected intermediate the storage tank and a fuel port of the air-fuel mixing chamber, wherein air from the air mover and fuel from the storage tank are mixed in the air-fuel mixing chamber and discharged through the outlet of the air-fuel mixing chamber to the inlet of the gas chamber, wherein an air-fuel mixture in the catalyst compartment combusts in the presence of the catalyst member to produce combustion products and heat, wherein the combustion products move through the tortuous pathway to the exhaust port, and wherein heat transferred, from the gas chamber to the fluid warming, surface of the biocompatible fluid warming chamber warms a flow of biocompatible fluids flowing through the biocompatible fluid warming chamber. The embodiment of the apparatus may comprise a catalyst member comprising, one of palladium and platinum. An embodiment of the apparatus may comprise a tank wherein the fuel stored in the tank is a hydrocarbon. An embodiment of the apparatus may comprise a heat exchanger base comprising a metal alloy. An embodiment of the apparatus may comprise a heat exchanger base comprising aluminum. An embodiment of the apparatus may comprise a fluid warming chamber wherein the fluid warmed in the fluid warming chamber is one of blood and intravenous fluid. An embodiment of the apparatus may comprise a fuel cell configured to receive a flow of fuel gas and to generate an electrical current to operate a motor within the air mover. An embodiment of the apparatus may comprise a valve that is adjustable to vary a rate of flow of fuel from the storage tank to the air-fuel mixing chamber. An embodiment of the apparatus may comprise a warming chamber wherein the warming surface of the fluid warming chamber comprises an undulating surface to increase the surface area across which heat can be received from the gas chamber and transferred to the fluid within the fluid warming chamber.

An embodiment of the apparatus of the present invention may further comprise a control system. For example, the apparatus may further comprise a controller coupled to receive a signal corresponding to an operating set-point input by a user of the apparatus wherein the controller generates and sends a signal to at least one of the motorized fuel valve and the air mover to adjust at least one of the rate of fuel and the rate of air delivered to the air-fuel mixing chamber.

### BRIEF DESCRIPTION OF THE DRAWINGS

FIG. 1 is a perspective view of an embodiment of a biocompatible fluid warming apparatus of the present invention.

FIG. 2 is an exploded perspective view of the biocompatible fluid warming apparatus of FIG. 1.

FIG. 3 is another exploded perspective view of the biocompatible fluid warming apparatus of FIG. 1.

FIG. 4 is a perspective view of a fuel assembly that can be used with the embodiment of the biocompatible fluid warming apparatus of FIGS. 1-3.

FIG. 5 is an elevation view of the outlet of an air mover of an embodiment of the apparatus of the present invention.

FIG. 6 illustrates a control system that can be used for an embodiment of the apparatus of the present invention.

### DETAILED DESCRIPTION OF PREFERRED EMBODIMENTS

An embodiment of the biocompatible fluid warming apparatus of the present invention provides a reduced overall volume of the apparatus and a corresponding increased power density in terms of the amount of heat transfer per unit volume. An embodiment of the biocompatible fluid warming apparatus of the present invention provides an improved air-fuel mixing chamber and a tortuous combustion products pathway within the apparatus to promote efficient transfer of heat from the combustion products moving through the combustion products pathway to the fluid to be warmed and introduced into a patient's body.

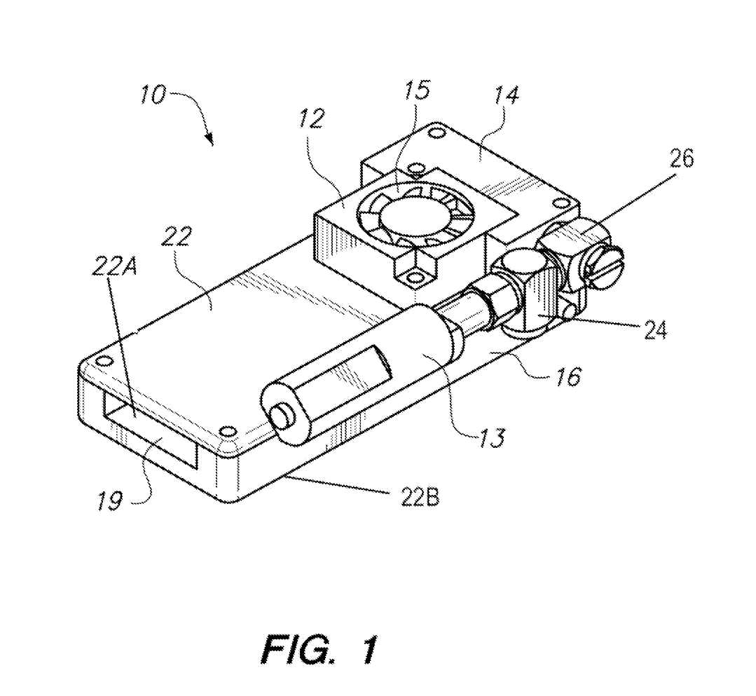

FIG. 1 is a perspective view of a portion 10 of an embodiment of a biocompatible fluid warming apparatus of the present invention. The portion 10 of the apparatus illustrated in FIG. 1 comprises a gas chamber cover 22 coupled to a heat exchanger base 16, an air mover 12, an air-fuel mixing chamber 14 coupled to the gas chamber cover 22, and a fuel assembly 13 coupled to the air-fuel mixing chamber 14. It should be noted that instead of the space-consuming venturi pathway common in conventional biocompatible fluid warming apparatuses, embodiments of the biocompatible fluid warming apparatus of the present invention include a compact air mover 12 such as, for example, a centrifugal fan, to receive and to move a stream of ambient air to an air-fuel mixing chamber 14.

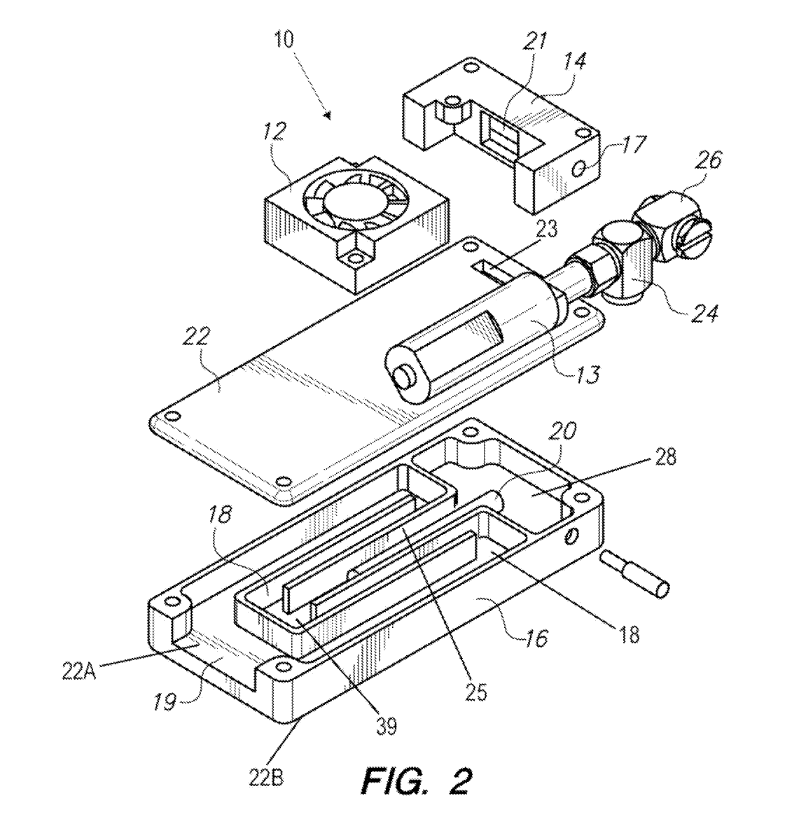

The embodiment of the heat exchanger base 16 illustrated in FIG. 1 and further illustrated in FIG. 2 comprises a gas chamber 39 having a receiving space 28, a catalyst compartment 25 and two tortuous pathways 18 originating at the catalyst compartment 25 and terminating at an exhaust port 19. A catalyst member 20 comprising a catalytic material that promotes combustion of the air-fuel mixture is disposed in the catalyst compartment 25 of the gas chamber 39 of the heat exchanger base 16. The catalyst material may be, for example, palladium or platinum. A pre-warmed air-fuel mixture enters the receiving space 28 of the gas chamber 39 of the heat exchanger base .16 through the gas chamber inlet 23 and moves through the catalyst compartment 25 and along the catalyst member 20. The catalyst member 20 promotes reaction of the pre-warmed air-fuel mixture to combustion gases to liberate heat. it will be understood that the composition of the combustion products depends on the fuel and will commonly include carbon dioxide and water vapor.

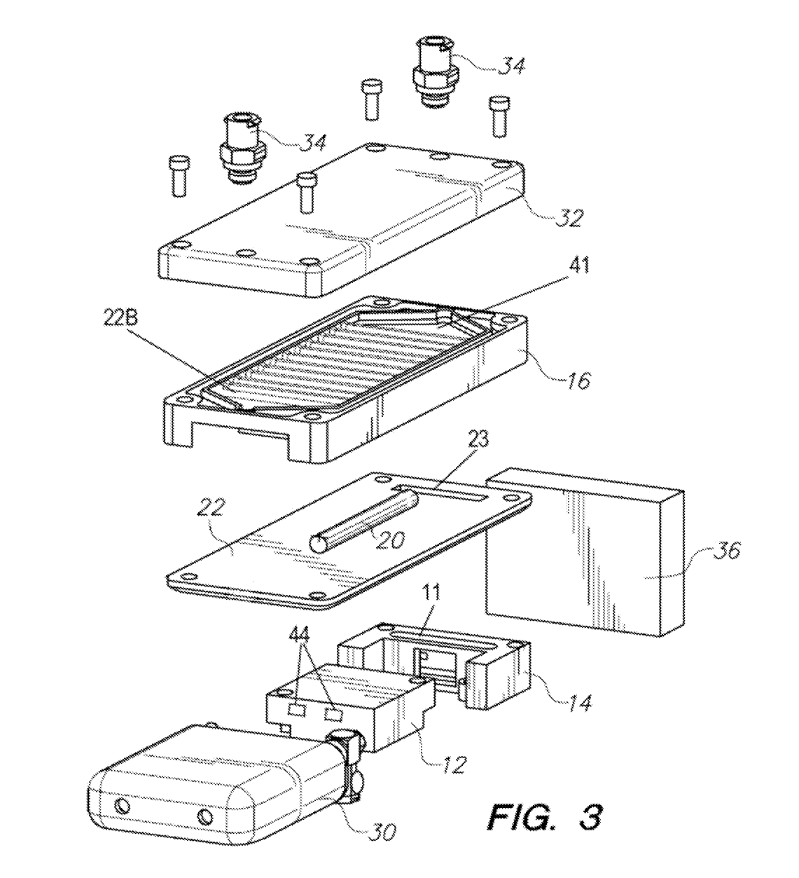

The hot combustion gases created by catalytic combustion of the air-fuel mixture in the catalyst compartment 25 move to and through the tortuous pathways 18 to the exhaust port 19 where they are liberated to the atmosphere. it will be understood that the heat generated by the combustion of the air-fuel mixture is transferred across the heat exchanger base 16 from a first side 22A (illustrated in FIGS. 1 and 2) to a second side 22B (illustrated in FIG. 3). It will be further understood that the tortuous pathways 18 are adapted to increase residence time of the hot combustion gases within the gas chamber 39 and downstream of the catalyst compartment 25 to thereby increase the amount of heat transferred to a fluid in the fluid warming chamber described in connection with FIG. 3.

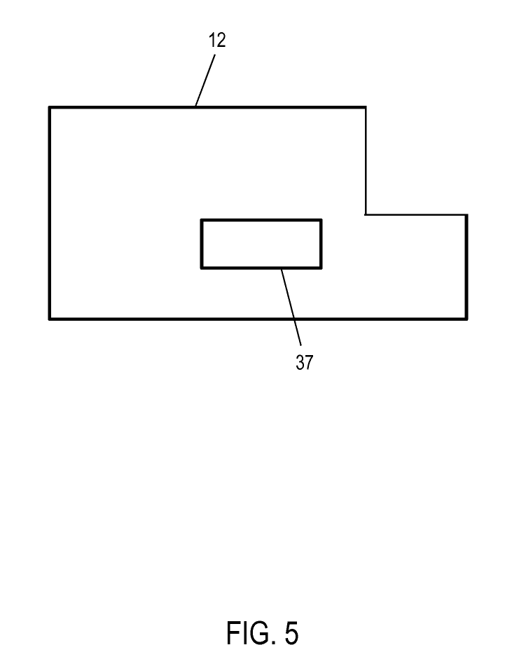

The air mover 12 illustrated in the coupled configuration in FIG. 1 is illustrated as removed from the was chamber cover 22 in FIG. 2. The air mover 12 discharges air through an outlet (shown in FIG. 5) of the air mover 14 to the inlet 21 of the air-fuel mixing chamber 14, it will be noted that in the assembled view of the portion 10 of the apparatus shown in FIG. 1, the air mover 12 is coupled to the gas chamber cover 22 immediately adjacent to the air-fuel mixing chamber 14 to sealably engage the outlet (shown in FIG. 5) of the air mover 12 with the intake 21 of the air-fuel mixing chamber 14 (shown in FIG. 2). The air-fuel mixing chamber 14 also receives a stream of fuel from a compressed fuel storage tank 13 through a fuel port 17 (shown in FIG. 2). In the embodiment of Wis. 1 and 2, compressed fuel gas or pressurized liquid fuel stored in the tank 13 is controllably throttled and/or released across a motorized needle valve 24 connected intermediate the tank 13 and a precision fuel delivery orifice 26 that is coupled to the fuel port 17 of the air-fuel mixing chamber 14. It will be understood that, although the embodiment of the apparatus of the present invention illustrated in the appended drawings comprises a motorized needle valve 24, a manually adjustable needle valve can also be used in alternate embodiments of the apparatus. It will be understood that the air stream and the fuel stream separately introduced into the air-fuel mixing chamber 14 through the air inlet 21 and the fuel port 17, respectively, are mixed within the air-fuel mixing chamber 14 by movement of the air stream discharged from the air mover 12 and by the movement of the fuel stream as it enters the air-fuel mixing chamber 14. In the assembled configuration of the portion 10 of the apparatus of FIG. 1, the air-fuel mixing chamber 14 is coupled to the gas chamber cover 22 proximal to the gas chamber inlet 23 to provide for pre-warming of an air-fuel mixture stream emerging from the outlet 11 (see FIG. 3) of the air-fuel mixing chamber 14. Pre-warming the air-fuel mixture results in a greater operating temperature in the heat exchanger base 16 and an increased heat exchange efficiency of the apparatus.

As can be seen in FIG. 3, the air-fuel mixture emerges from the outlet 11 of the air-fuel mixing chamber 14 and enters the ₕas chamber 39 (not shown in FIG. 3—see FIG. 2) between the heat exchanger base 16 and the gas chamber cover 22 through the heat exchanger base inlet 23 of the gas chamber cover 22. The catalyst member 20, shown in FIG. 2 in the catalyst compartment 25 and in FIG. 3 removed from the catalyst compartment 25, is elongate and is resides in the elongate catalyst compartment 25 of the gas chamber 39. The tortuous combustion product pathways 18 originate at the catalyst compartment 25 and terminate at the exhaust port 19 at an end of the heat exchanger base 16. The embodiment of the heat exchanger base 16 illustrated in FIG. 2 illustrates a configuration of two separate pathways 18 for moving combustion products from the catalyst compartment 25 to the exhaust port 19, each pathway 18 having switchbacks to provide for increased residence time within the gas chamber 39 of the hot combustion products gases emerging from the catalyst compartment 25. The increased residence time results in improved overall heat transfer efficiency. It should be noted that the catalyst member 20 can be selectively positioned proximally (closer to the receiving space 28) or distally (closer to the exhaust port 19) within the catalyst compartment 25 for “fine-tuning” of air-fuel mixing occurring upstream of the catalyst compartment 25 and for optimization of the catalytic combustion efficiency.

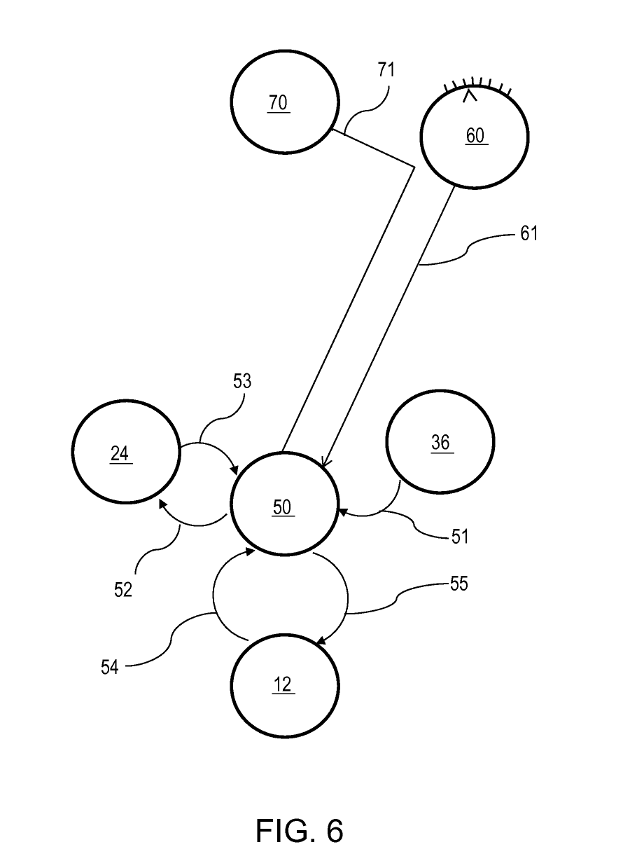

The biocompatible fluid warming apparatus illustrated in the appended drawings includes a generally flat and rectangular heat exchanger base 16, but this particular design aspect is not crucial to the function. Alternatively, a cylindrical heat exchanger base as disclosed in U.S. Pat. No. 7,261,537 may be used. Alternately, the gas chamber cover 22 ma comprise a catalyst to supplement or complement the catalyst member 20. It will be understood that the motorized needle valve 24 and the precision fuel delivery orifice 26 may be either manually or automatically adjusted and/or modified to optimize the rate of fuel flow to the air-fuel mixing chamber 14. Control of the operation of apparatus will be discussed in connection with FIG. 6.

FIG. 3 illustrates the heat exchanger base 16 and gas chamber cover 22, air-fuel mixing chamber 14, air mover 12, motorized needle valve 24, precision fuel delivery orifice 26, fluid warming chamber 41, fluid warming chamber cover 32 and a pair of Luer lock fittings 34 coupled to the fluid warming chamber cover 32. A battery pack 36 is provided to supply electrical current to a motor (not shown) incorporated within the air mover 12 or, alternately, a fuel cell 30 engages the air mover 12 through electrical contacts 44 to provide electrical current to operate the air mover 12 and, optionally, to operate the motorized needle valve 24. It will be understood that a variety of fuels may be stored in the tank 13 and used to fuel the catalytic combustion such as, for example, butane or propane.

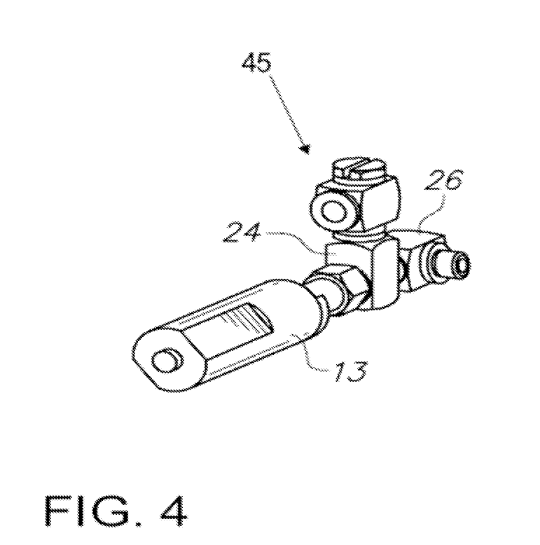

FIG. 4 is a view of an embodiment of the fuel assembly 45 comprising the tank 13, the motorized needle valve 24 and the precision fuel delivery orifice 26.

FIG. 5 is an elevation view of the outlet 37 of an air mover 12 of an embodiment of the apparatus of the present invention. The outlet 37 delivers air discharged from the air mover 12 into the inlet 21 of the air-fuel mixing chamber 14. It will be understood that a seal may be provided about the outlet 37 of the air mover 12 and/or about the inlet 21 of the air-fuel mixing chamber 14.

FIG. 6 illustrates a control system that can be used for an embodiment of the apparatus of the present invention. The air mover 12 and the motorized needle valve 24 are shown along with the battery 36 as components that interact through a controller 50. The controller 50 may be electronically coupled to receive an operating set-point signal 61 entered on an input instrument which may comprise a dial, keypad, button, switch, slide, etc. The controller 50 reads the operating set-point signal 61 and compares it to a valve position signal 53 that indicates the amount of fuel being provided to the air-fuel mixing chamber 14 (not shown in FIG, 6). The controller 50 may automatically adjust the position of the motorized needle valve 24 by generating and sending a signal 52 to the motorized needle valve 24 corresponding, to the operating set-point signal 61. The controller 50 may further generate and send a signal 55, such as an electrical current, to the air mover 12 to adjust the throughput of the air mover 12 to correspond to the adjusted fuel rate provided by the adjustment of the position of the motorized needle valve 24.

In an alternate control scheme, the controller 59 reads the operating set-point signal 61 and compares it to an air mover throughput signal 54 that indicates the amount of air being moved through the air mover 12 to burn the fuel being provided to the air-fuel mixing chamber .14 (not shown in FIG. 6). The controller 50 may adjust the throughput of the air mover 12 by generating and sending a signal 55, such as an electrical current, to the air mover 12 corresponding to the operating set-point signal 61. The controller 50 may further generate and send a signal 52 to adjust the position of the motorized needle valve 24 to adjust the rate of fuel delivered to the air-fuel mixing chamber 14 to match the fuel delivery rate through the motorized needle valve 24 to correspond to the adjusted air mover throughput. In a related embodiment, a temperature sensor 70 may be used to generate and send a signal 71 indicating the temperature of the fluid leaving the fluid warming chamber 41, either continuously or periodically, to the controller 50 for comparison to an operating, set-point 61. The controller 50 may be programmed to adjust the position of the motorized needle valve 24, the throughput of the air mover 12, or both, to bring the temperature of the fluid leaving the warming chamber 41 and the corresponding signal 71 close to the operating set-point 61. Embodiments of the apparatus of the present invention having a system for enabling controller 50 monitoring and/or control of the position of the valve 24 that controls the rate of flow of fuel to the air-fuel mixing chamber 14 and/or the throughput of the air mover 12 can be used to optimize the air/fuel ratio within the catalyst compartment 25 and thereby conserve both fuel and battery life.

The interior surfaces of the gas chamber 39 and/or the fluid warming chamber 41 may include undulations, ridges, channels or other features that increase the overall surface area of the gas chamber 39 and/or the fluid warming chamber 41 to promote increased heat transfer from the first side 22A of the heat exchanger base 16 to the second side 22B of the heat exchanger base 16. The interior surfaces of the fluid warming chamber cover 32 and the gas chamber cover 22 may be coated, treated and/or without undulations, ridges, channels or other features that increase the overall surface area of the fluid warming chamber cover 32 and the gas chamber cover 22 in order to minimize heat transfer from the gas chamber 39 to a component of the apparatus other than the heat exchanger base 16 across which heat is conducted to the fluid warming chamber 41.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, components and/or groups, but do not preclude the presence or addition of one or more other features, integers, steps, operations, elements, components, and/or groups thereof. The terms “preferably,” “preferred,” “prefer,” “optionally,” “may,” and similar terms are used to indicate that an item, condition or step being referred to is an optional (not required) feature of the invention.

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. The embodiments described herein were chosen and described in order to best explain the principles of the invention and the practical application, and to enable others of ordinary skill in the art to understand the invention for various embodiments with various modifications as are suited to the particular use contemplated. Although specific embodiments of the invention have been described herein in some detail, this has been done solely for the purposes of explaining, the various aspects of the invention, and is not intended to limit the scope of the invention as defined in the claims which follow.

We claim:

1. An apparatus, comprising:
a gas flow chamber on a first side of the apparatus having an air-fuel mixture inlet, a catalyst compartment and at least one tortuous combustion products pathway originating at the catalyst compartment and terminating at an exhaust gas port;
a fluid warming chamber on a second side of the apparatus to conductively receive heat generated in the gas flow chamber and having, a fluid inlet connectable to a source of fluid, a fluid warming surface and a fluid outlet connectable to a patient;
an air-fuel mixing chamber having an air inlet, a fuel port and an air-fuel mixture a motor-driven air mover having an air intake to receive ambient air and an air outlet disposed to discharge air to the air inlet of the air-fuel mixing chamber; and
a fuel assembly comprising a fuel storage tank, a valve to receive a stream of fuel from the tank and a fuel port connector coupled to provide fuel from the valve to the fuel port of the air-fuel mixing chamber;
wherein a stream of an air-fuel mixture emerging from the air-fuel mixing chamber enters the catalytic compartment containing the catalyst member and combusts to create a stream of heated combustion products;
wherein the combustion products flow through the at least one tortuous pathway to the exhaust port where the combustion products are liberated to the atmosphere; and
wherein a stream of fluid from the source of fluid enters the fluid warming chamber through the fluid inlet, is warmed along the warming surface and is removed from the fluid warming chamber through the fluid outlet.

2. The apparatus of claim 1, wherein the catalyst member comprises one of palladium and platinum.

3. The apparatus of claim 1, wherein the fuel stored in the tank is a hydrocarbon.

4. The apparatus of claim 1, wherein the heat exchanger base comprises a metal alloy.

5. The apparatus of claim 4, wherein the heat exchanger base comprises aluminum.

6. The apparatus of claim 1, wherein the fluid warmed in the fluid warming chamber is one of blood and intravenous fluid.

7. The apparatus of claim 1, further comprising:
a fuel cell configured to receive a flow of fuel gas and to generate an electrical current to operate a motor within the air mover.

8. The apparatus of claim 1, wherein the valve is adjustable to vary a rate of flow of fuel from the storage tank to the air-fuel mixing chamber.

9. The apparatus of claim 1, wherein the warming surface of the fluid warming chamber comprises an undulating surface to increase the surface area across which heat can be received from the gas chamber and transferred to the fluid within the fluid warming chamber.

10. An apparatus, comprising:
a heat exchanger base having a first side and a second side;
a gas chamber cover securable to the first side of the heat exchanger base to form a gas chamber therebetween, the gas chamber having an inlet, a catalyst compartment, a tortuous pathway and an exhaust port;
a biocompatible fluid warming chamber cover securable to the second side of the heat exchanger base to form a fluid chamber therebetween, the biocompatible fluid warming chamber having an inlet connectable to a source of biocompatible fluid, an outlet connectable to a patient, and a fluid warming surface therebetween;
an air-fuel mixing chamber having an outlet sealably engaging the inlet to the gas chamber;
a catalyst member disposed within the catalyst compartment of the gas chamber, an air mover having an ambient air inlet and an air outlet sealably engaging an air intake of the air-fuel mixing chamber;
a storage tank containing a fuel; and
a valve connected intermediate the storage tank and a fuel port of the air-fuel mixing chamber;
wherein air from the air mover and fuel from the storage tank are mixed in the air-fuel mixing chamber and discharged through the outlet of the air-fuel mixing chamber to the inlet of the gas chamber;
wherein an air-fuel mixture in the catalyst compartment combusts in the presence of the catalyst member to produce combustion products and heat;
wherein the combustion products move through the tortuous pathway to the exhaust port; and
wherein heat transferred from the gas chamber to the fluid warming surface of the biocompatible fluid warming chamber warms a flow of biocompatible fluids flowing through the biocompatible fluid warming chamber.

11. The apparatus of claim 10, wherein the catalyst member comprises one of palladium and platinum.

12. The apparatus of claim 10, wherein the fuel stored in the tank is a hydrocarbon.

13. The apparatus of claim 10, wherein the heat exchanger base comprises a metal alloy.

14. The apparatus of claim 13, wherein the heat exchanger base comprises aluminum.

15. The apparatus of claim 10, wherein the fluid warmed in the fluid warming chamber is one of blood and intravenous fluid.

16. The apparatus of claim 10, further comprising:
a fuel cell configured to receive a flow of fuel gas and to generate an electrical current to operate a motor within the air mover.

17. The apparatus of claim 10, wherein the valve is adjustable to vary a rate of flow of fuel from the storage tank to the air-fuel mixing chamber.

18. The apparatus of claim 10, wherein the warming surface of the fluid warming chamber comprises an undulating surface to increase the surface area across which heat can be received from the gas chamber and transferred to the fluid within the fluid warming chamber.

19. The apparatus of claim 1, further comprising:
a controller coupled to receive a signal corresponding to an operating setpoint input by a user of the apparatus;
wherein the controller generates and sends a signal to at least one of the motorized fuel valve and the air mover to adjust at least One of the rate of fuel and the rate of air delivered to the air-fuel mixing chamber.

20. The apparatus of claim 10, further comprising:
a controller coupled to receive a signal corresponding to an operating set-point input by a user of the apparatus;
wherein the controller generates and sends a signal to at least one of the motorized fuel valve and the air mover to adjust at least one of the rate of fuel and the rate of air delivered to the air-fuel mixing chamber.
